(19) United States
(12) Patent Application Publication
(10) Pub. No.: US 2009/0036540 A1
(43) Pub. Date: Feb. 5, 2009

(54) Guanylhydrazones in Methods of Treatment or Diagnosis as Modulators of Signal Transduction

(75) Inventors: Sander H. Diks, 's Hertogenbosch (NL); Klaartje Kok, Utrecht (NL); Maikel P. Peppelenbosch, Utrecht (NL)

Correspondence Address: Law Office of John K. Pike, PLLC, 2121 Eisenhower Avenue, Suite 200, Alexandria, VA, 22314, US

(73) Assignee: Cytokine PharmaSciences, Inc., King of Prussia, PA (US)

(21) Appl. No.: 12/252,340

(22) Filed: Oct. 15, 2008

Related U.S. Application Data
(63) Continuation of application No. 10/987,110, filed on Nov. 15, 2004, now abandoned.
(60) Provisional application No. 60/519,633, filed on Nov. 14, 2003.

(51) Int. Cl. A61K 31/155; C12N 5/08; A61P 35/00; A61P 25/00; A61P 9/00

(52) U.S. Cl. 514/632; 435/366

(57) ABSTRACT

A method is provided for the treatment of a condition mediated by raf kinase, which includes administering a guanylhydrazone to a subject in need thereof. A method is also provided, which includes modulating or inhibiting signal transduction in a c-raf pathway with at least one guanylhydrazone. Another method is provided, which includes contacting one or more human mononuclear cells with at least one guanylhydrazone and at least one lipopolysaccharide to obtain one or more treated cells; contacting at least one selected from the group including said treated cells, one or more lysates thereof, and combinations thereof, with at least one surface-bound peptide in a surface-bound peptide array; and selectively modulating or inhibiting the phosphorylation of the surface-bound peptide.

### CROSS-REFERENCE TO RELATED APPLICATIONS

This application claims priority from U.S. Provisional Application Ser. No. 60/519,633, filed Nov. 14, 2003. The entirety of that provisional application is incorporated herein by reference.

### BACKGROUND OF THE INVENTION

1. Field of the Invention

The invention is directed to methods of treatment and diagnosis using a guanylhydrazone as a modulator of kinase signal transduction.

2. Discussion of the Background

The DNA array technique allows the analysis of the genome and transcriptome of cells and has revolutionized the study of molecular biology. Massive parallel analysis using DNA array technology has become the mainstay for the analysis of genomes and transcriptomes. (Conway, T. & Schoolnik, G. K., and Wurmbach, E. et al.)

Extensive analyses (Velculescu, et al.) provide evidence that there are differences in transcriptomes of different cell types. The majority of this transcriptome, however, is necessary to keep a cell functioning and could be regarded as the minimal transcriptome. Only a small portion of the transcripts present in the cell determines cell fate and these critical transcripts are expressed at low levels. Only a small amount of transcripts are necessary to convert a normal cell to another as evidenced by the small differences between SAGE banks of cells from two different organs. Accordingly, small changes in expression levels can lead to large changes in enzyme activity and subsequently significant alterations in cell functioning. Thus, a comprehensive description of cellular metabolism may be more useful than such a description of the transcriptome.

Currently available technologies focus on the static determination of the relative concentration of metabolites but does not address the actual activity of various cellular signaling pathways. Accordingly, no current techniques are in existence that allow a likewise parallel assessment of the intracellular biochemistry of cells. Array technology has not been used for measuring enzymatic activity in cellular lysates.

The kinome, the specific activity of different kinases present in a cell, of peripheral blood-derived human mononuclear cells before and after stimulation with lipopolysaccharide (LPS) can be studied using cell lysates and in vitro phosphorylation of arrays.

U.S. Pat. No. 5,599,984 is directed to guanylhydrazones and their uses in treating inflammatory conditions. The guanylhydrazone compound known as Semapimod is disclosed therein as compound number fourteen.

U.S. Patent Publication No. 2003-0181442 A1 is directed to compounds and therapies used in treating Raf mediated diseases.

U.S. Patent Publication No. 2003-0153588 A1 is directed to compounds and therapies used in treating Raf mediated diseases.

U.S. Patent Publication No. 2003-0144278 A1 is directed to compounds and therapies used in treating Raf mediated diseases.

U.S. Patent Publication No. 2003-0139605 A1 is directed to compounds and therapies used in treating Raf mediated diseases.

U.S. Patent Publication No. 2003-0134837 A1 is directed to compounds and therapies used in treating Raf mediated diseases.

U.S. Patent Publication No. 2001-0006975 A1 is directed to compounds and therapies used in treating Raf mediated diseases.

Kolodnev et al., Cancer Research, 63, 18, 5669-5673 (Sep. 15, 2003). The article is directed to compounds and therapies used in treating Raf mediated diseases.

Choi et al., Proc. Am. Assoc. Canc. Res., 44, p. 138, (July 2003). The article is directed to compounds and therapies used in treating Raf mediated diseases.

### SUMMARY OF THE INVENTION

The above problems, and others, are solved by the present invention.

One embodiment of the present invention provides a method for the treatment of a condition mediated by raf kinase, which includes administering a guanylhydrazone to a subject in need thereof.

Another embodiment of the present invention provides a method, which includes modulating or inhibiting signal transduction in a c-raf pathway with at least one guanylhydrazone.

Another embodiment of the present invention provides a method, which includes:

contacting one or more human mononuclear cells with at least one guanylhydrazone and at least one lipopolysaccharide to obtain one or more treated cells;

contacting at least one selected from the group including the treated cells, one or more lysates thereof, and combinations thereof, with at least one surface-bound peptide in a surface-bound peptide array; and

selectively modulating or inhibiting the phosphorylation of the surface-bound peptide.

### DESCRIPTION OF THE FIGURES

The foregoing description will be better understood when read in conjunction with the appended drawings. For the purpose of illustrating the invention, there is shown in the drawings an embodiment which is presently preferred, it being understood, however, that this invention is not limited to the precise arrangements and instrumentalities shown.

FIG. 1 is a graphical depiction of a peptide array demonstrating detection using phospho-imaging of a consensus peptide sequence of protein kinase A. Layout of the Trial PepChip containing 2*192 pseudo substrates. The 192 pseudo substrates are grouped in 8 clusters of 24 spots as depicted in the upper right layout. In the upper left panel an phosphorylation profile of Protein Kinase A is shown. The pseudo peptide spots marked with an asterisk have been shown in the lower panel and a Protein Kinase A phosphorylation consensus sequence has been extrapolated from the phosphorylated pseudo substrates.

FIG. 2 is a graphical depiction of a peptide array demonstrating detection of lysates from human PBMCs stimulated with LPS. Detection is done using phospho-imaging of a consensus peptide sequence for MAP kinase family members. Peripheral Blood Mononuclear Cells were stimulated with lipopolysaccharide and a kinome array has been produced using the Trial PepChip. The upper two plots are weighted average profiles of four values. The lower plot is an overlay in which green dots are LPS up-regulated pseudo substrates, red dots are LPS down-regulated pseudo substrates and yellow dots are substrates that are not altered by LPS stimulation.

FIG. 3 is a graphical of a peptide array demonstrating detection of lysates. The dot plots are example blow ups of one 24-dot-cluster. The left panel in the unstimulated situation, the middle represents the LPS stimulated situation and the right panel the LPS stimulated situation with a semapimod pre-incubation In the lower part of the figure are overlay plots displayed for LPS specificity and semapimod sensitivity.

FIG. 4 is a graphical depiction of a Western blot analysis confirming that Semapimod abolishes MEK phosphorylation in PBMCs. Western blots were performed on PBMCs to detect phosphorylated proteins upon LPS stimulation. The left part of the graph is the unstimulated situation and the right part is the stimulated situation. The PBMCs were probed for phosphorylated MAP kinases (p38, p42/44 and JNK), upstream kinases (MEK1/2 and c-Raf), Akt/PKB and PKC-ζ.

FIG. 5 is a graphical depiction of a Western blot analysis showing the effect of semapimod on LPS-dependent phosphorylation of c-Raf. MEK1/2 was used as a control to determine the specificity of the phosphorylation of the pseudo substrates.

FIG. 6 is a graphical depiction of a Western blot analysis. Reduced pP38, pJnk, perk in 4/4 macrophages treated with Semapimod (1 and 0.1 μM) in LPS present conditions was seen. The phosphorylation status of MKK4/7 (upstream of Jnk), and Mek1/2 (upstream of Erk) was affected due to Semapimod treatment. pRaf expression was not influenced by Semapimod.

FIG. 7 is a graphical depiction of a Western blot analysis. In vitro kinase assay showed reduced pMek1/2 expression upon 5 and 10 minute treatment with 1 μM Semapimod. Pan-Raf protein expression was analyzed to test for equal loading.

FIG. 8 shows the immunohistochemical staining of biopsies obtained from Crohn's disease patients who participated in the Semapimod study. The data is shown for responders and non-responders for A—before Semapimod treatment and B—after Semapimod treatment. Immunohistochemical stainings are shown for responder patients 4, 6, 9, 10 and 11, and non-responder patient The immunohistochemical staining of biopsies obtained from responders (n=5) demonstrates a significant decrease (p=0.0348) of pMek1/2 expression upon Semapimod treatment.

FIG. 9A shows a part of the IHC analysis of Crohn's disease patients who participated in the Semapimod pilot study. More particularly, FIG. 9A shows the pMek expression before and after CNI-treatment in these patients. Very little pMek1/2 expression was seen at baseline in patient 004.

FIG. 9B shows another part of the IHC analysis of Crohn's disease patients who participated in the Semapimod pilot study. FIG. 9B generally shows the CRP serum levels of these patients. CRP levels of patient #12 were not affected by Semapimod (e.g. nonresponder), and pMek1/2 expression was also not affected by Semapimod in this nonresponder. The finding that very little pMek1/2 expression was seen at baseline in patient 004 (e.g., in FIG. 9A) correlates with the low CRP levels at baseline (e.g., in FIG. 9B).

### DETAILED DESCRIPTION OF THE INVENTION

Various other objects, features, and attendant advantages of the present invention will be more fully appreciated as the same becomes better understood from the following detailed description of the invention, which is not intended to be limiting unless otherwise specified.

The invention relates to the discovery that guanylhydrazones, and in particular, Semapimod, modulates or inhibits signal transduction, and particularly along the c-Raf pathway. It has also been discovered that various diseases and conditions associated with the c-Raf pathway can be modulated or inhibited, based on the inhibitory effect that guanylhydrazones have upon signal transduction along this kinase pathway.

Accordingly, in a preferred embodiment, the present invention relates to methods of treatment and diagnosis using a guanylhydrazone as a modulator of kinase signal transduction. Another preferred embodiment of the present invention relates to the discovery that guanylhydrazones modulate or inhibit signal transduction through the c-Raf MEK kinase. Still other embodiments of the invention relate to methods of treatment or diagnosis by modulating signal transduction through the c-Raf MEK kinase using guanylhydrazone and preferably Semapimod.

The present inventors have found that the kinome, the specific activity of different kinases present in a cell, of peripheral blood-derived human mononuclear cells before and after stimulation with lipopolysaccharide (LPS) can be studied using cell lysates and in vitro phosphorylation of arrays.

By the present invention, it is possible to determine and understand the actual activity of various cellular signaling pathways. Another embodiment of the present invention relates to a method that enables a parallel assessment of the intracellular biochemistry of cells. Another embodiment of the present invention relates to a method for measuring enzymatic activity in cellular lysates using array technology.

In part by using cell lysates and in vitro phosphorylation of arrays which include 192 peptides with consensus sequences for different kinases spotted on glass, the present inventors have analyzed the kinome (the specific activity of different kinases present in a cell) of peripheral blood-derived human mononuclear cells before and after stimulation with lipopolysaccharide (LPS). Using this system, the present inventors have also studied the effects of Semapimod, an anti-inflammatory compound, on the kinome of LPS-stimulated cells. Semapimod has demonstrated remarkable clinical efficacy in steroid-refractory Crohn's disease, and one embodiment of the present invention relates to a method for better understanding its mechanism of action. According to one embodiment of the present invention, MEK kinases are a relevant target for understanding this mechanism better so using array technology is a useful strategy for analysing the relative activity of cellular metabolic pathways.

Surprisingly, employing array technology allows faster and more comprehensive analysis of cellular metabolism in comparison to all currently available technology, which focuses on the static determination of the relative concentration of metabolites but does not address the actual activity of various cellular signaling pathways. Hence, the usefulness of array technology for studying the cellular kinome (the combined activity of all cellular kinases) was tested.

Suitable guanylhydrazones are disclosed in, for example, U.S. Pat. No. 5,599,984, the entire contents of which are hereby incorporated by reference. Semapimod is disclosed therein as compound number fourteen. The guanylhydrazone and/or semapimod may be administered alone, or as a salt, or in combination. The compound may be administered as a composition in combination with one or more pharmaceutically acceptable carriers or diluents.

The present invention includes a method for treating raf-mediated diseases and conditions. Suitable raf-mediated diseases and conditions are disclosed in, for example, U.S. Patent Publication Nos. 2003-0181442 A1, 2003-0153588 A1, 2003-0144278 A1, 2003-0139605 A1, 2003-0134837 A1, 2001-0006975 A1; and Kolodney et al., Cancer Research, 63, 18, 5669-5673 (Sep. 15, 2003), and Choi et al., Proc. Am. Assoc. Canc. Res., 44, p. 138, (July 2003), the entire contents of which are hereby incorporated by reference.

In silico analysis of the phosphobase resource enabled identification of consensus amino acid phosphorylation sequences for most kinases present in mammalian genome. (Blom, N., Kreegipuu, A. & Brunak, S, and Kreegipuu, A., Blom, N. & Brunak, S.) Further analysis of this set of kinase substrates revealed that the mean amino acid residue length that achieves an optimal specificity/sensitivity ratio for substrate phosphorylation was nine-amino acids. Thus, for constructing an array nonapeptides were utilized. Arrays were constructed by chemically synthesizing soluble peptides, which were covalently coupled to glass substrates. Arrays included 192 different nonapeptides (denominated: A1-P12), providing kinase substrate consensus sequences across the entire mammalian kinome.

On each separate carrier, the array was spotted two times, to allow assessment of possible variability in substrate phosphorylation. The final physical dimensions of the array were 19.5×19.5 mm, each peptide spot having a diameter of approximately 350 μm, and peptide spots being 750 μm apart. Suitable peptide arrays are available, for example, from Pepscan Systems, Edelhertweg 15 8219 PH Lelystad, The Netherlands.

To set up an appropriate design, addition of purified kinases in the presence of ATP results in the phosphorylation of the appropriate consensus peptide sequences without concomitant phosphorylation of other peptides. To test the extent this actually occurs, the array was set for 2 hours at 30° C. with 50 ng of the constitutively active catalytic subunit of protein kinase A (PKA) and γ-³³P-labeled ATP. This treatment resulted in the phosphorylation of target peptides with ³³P, allowing detection using phospho-imaging.

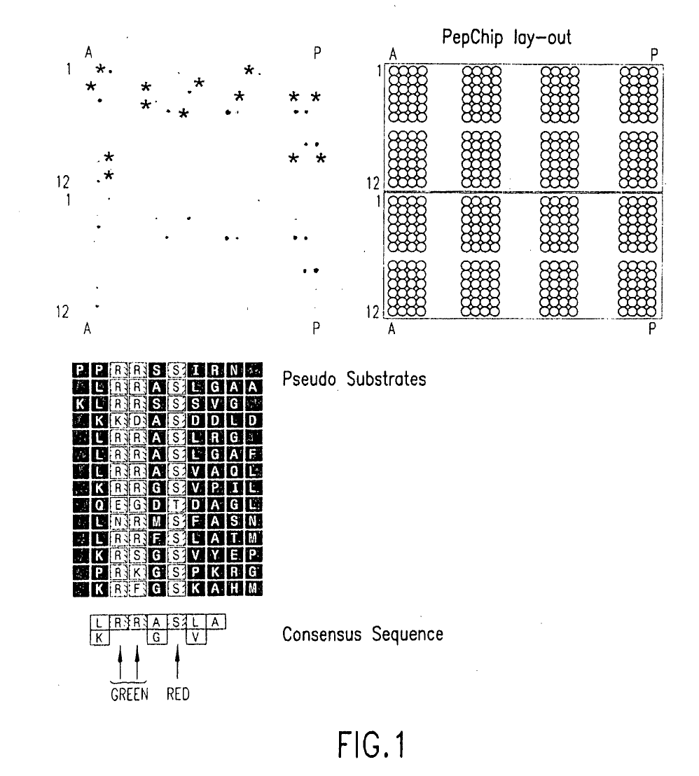

As depicted in FIG. 1, this showed a strong phosphorylation of the peptides on the array, which contained a PKA consensus sequence, whereas accompanying phosphorylation of peptides not containing PKA consensus phosphorylation sites was negligible. These results identified the array as useful for determining substrate specificity of kinases.

The present inventors have found that the peptide array is also useful for kinome profiling of actual whole cell lysates. To perform kinome profiling, human peripheral blood-derived mononuclear cells (PBMCs) were utilized. In contrast to permanent cell lines, these untransformed cells have very little kinase activity in unstimulated conditions. Lipopolysaccharide is a component of the cell wall of gram-negative bacteria and is a major activator of innate immune system, mediating for instance septic shock in human disease, and is well documented to elicit activation of a variety of kinases in human peripheral blood-derived mononuclear cells. (Morrison, D. C. & Ryan, J. L. and Weinstein, et al.) Hence lipopolysaccharide stimulation of the human peripheral blood-derived mononuclear cells was used as an attractive model for testing array-based kinome profiling.

To this end, lysates of human peripheral blood-derived mononuclear cells were treated for 15 minutes with vehicle or 100 ng/ml lps and were compared with respect to their capacity to phosphorylate peptides on arrays. Lysates from unstimulated cells produced only little phosphorylation of the immobilised peptides. Of the oligopeptides (7-9 amino acids) that were phosphorylated many were derived from consensus sites for cytoskeletal component-derived peptides (e.g. the p34cdc2-sites in vimentin, lamin B1, and caldesmon, the p37 kinase sites in vimentin), (Table 1) which reflects the highly motile phenotype of these cells. In addition peptides derived from enzymes implicated in basal cell metabolism (e.g. the casein kinase II site in DNA topoisomerase II and the phosphorylase kinase site in glycogen phosphorylase) were major substrates for cell lysates of these unstimulated cells. (Table 1). Thus the kinome profile obtained from human peripheral blood-derived mononuclear cells using in vitro phosphorylation of a peptide array is not inconsistent with the profile expected from resting monocytes.

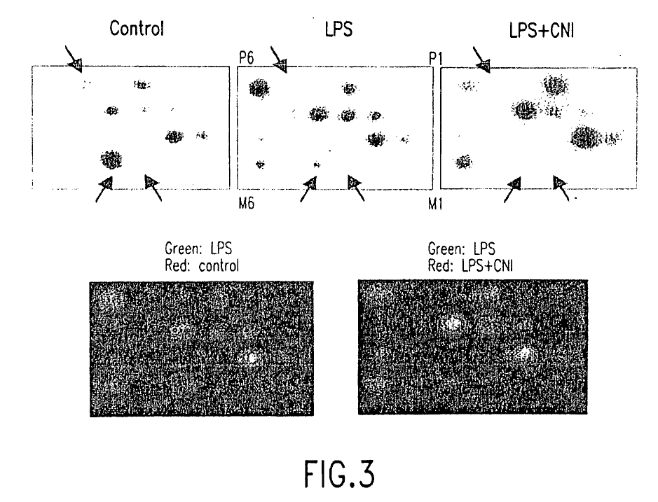

Subsequently the effect of incubating peptide arrays with lysates obtained from human peripheral blood-derived mononuclear cells stimulated with lipopolysaccharide was examined. This resulted in the specific incorporation of ³³P in a variety of peptides, suggesting that the lipopolysaccharide treatment activated the kinases for which these peptides represent a consensus phosphorylation sequence. The latter notion was confirmed by in parallel-performed SDS-PAGE followed by Western blotting of the cells and probing with phosphorylation-state specific antibodies. (FIG. 3)

In the array it was observed that lipopolysaccharide-dependent phosphorylation especially of peptides with consensus phosphorylation sequences for p38 MAP kinase (e.g. the p38 MAP kinase phosphorylation site of MAPKAP kinase 2, Heat Shock Protein 27); and the protein kinase Cζ (the protein kinase Cζ phosphorylation site in c-Src) (Table1).

Kinases that are able to phosphorylate p42/44 MAPK e.g. PKC-ζ and PKG are also upregulated upon LPS stimulation (Monick, et al.). Phosphorylation of peptides containing the tyrosine in STAT-1α/β that mediates the activation of this transcription factor, was highly upregulated upon LPS stimulation, in agreement with earlier studies (Ohmori, et al.) In addition, phosphorylation of peptides derived from cytoskeletal proteins became even more pronounced as in unstimulated cells (e.g. the casein kinase II site in DNA topoisomerase II and the phosphorylase kinase site in glycogen phosphorylase), in agreement with the effects of LPS on cell morphology and endocytosis. The experiments were performed with cells stimulated with LPS for 15 min, a time frame in which the activity of various LPS-induced kinases are already downregulated, which may explain the lack of phosphorylation of Calmoldulin-II (CaM-11)-derived peptides. Thus, with the present invention, it is possible to perform kinome profiling in cellular lysates using in vitro phosphorylation of peptides arrays.

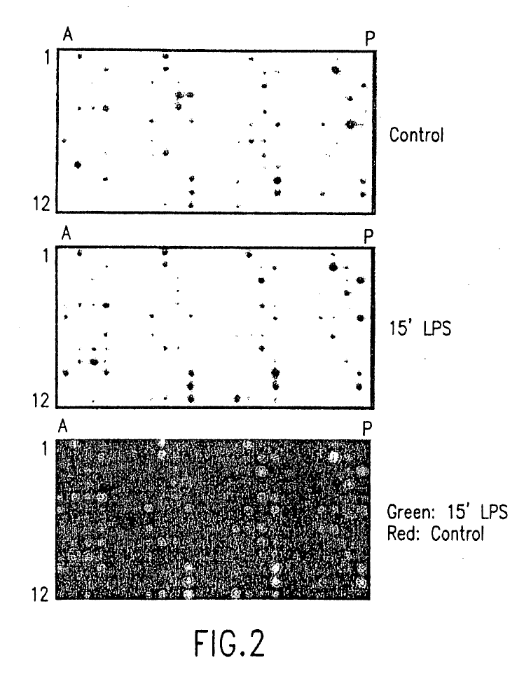

As evident from FIG. 2, the lipopolysaccharide treatment activated all of these kinases as judged by western blot analysis performed in parallel. In addition, phosphorylation of peptides derived from cytoskeletal proteins became even more pronounced as in unstimulated cells (e.g. the casein kinase II site in DNA topoisomerase H and the phosphorylase kinase site in glycogen phosphorylase), in agreement with the effects of LPS on cell morphology and endocytosis (Peppelenbosch blood). It was concluded that it is possible to perform kinome profiling using peptide arrays.

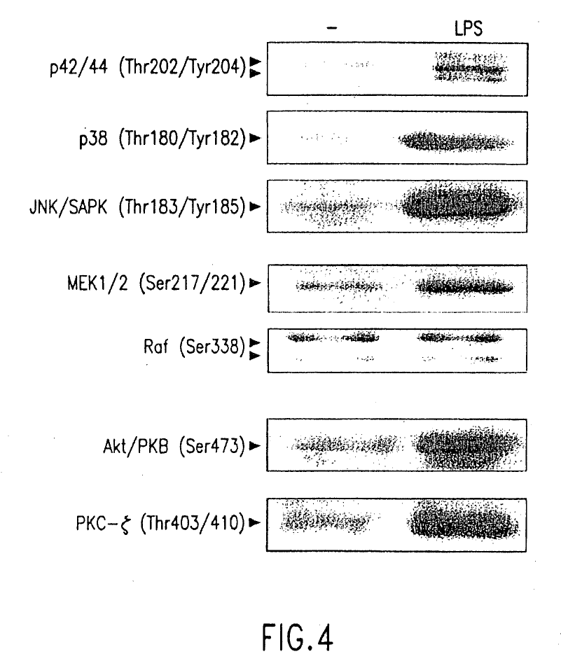

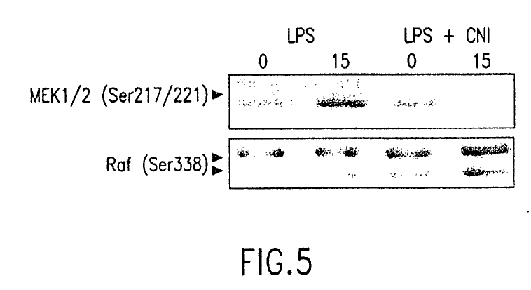

The peptide arrays are a valid tool for describing the kinome. In addition, the present inventors have found that the technique enables the determination of the molecular targets of kinase inhibitors. The present technique also allows one to identify the proteins mediating the effect of Semapimod, a potent and high-profile compound that induces clinical remission and endoscopal healing in steroid- and infliximab-resistant Crohn's disease. (Hommes, D. et al.) Although semipimod is generally considered to inhibit MAP kinase activation of immune cells (Hommes, D. et al. and Cohen, P. S. et al.), until the present invention, a molecular target has not been identified as yet. In the analysis, the kinase activity profile of lysates from human peripheral blood-derived mononuclear cells stimulated with lipopolysaccharide in the presence or absence of Semapimod were analyzed. Lysates obtained from Semapimod-treated cell of cells were strongly impaired in their capacity to phosphorylate peptides representing consensus sequences for MAP kinase family members (FIG. 2) (Table 1). Phosphorylation of a peptide representing a consensus phosphorylation site for MEK kinase was inhibited as well and Western blot analysis confirmed that Semapimod treatment abolishes MEK phosphorylation in human peripheral blood-derived mononuclear cells (FIG. 4). The phosphorylation of a peptide representing the PKC consensus phosphorylation site in c-Raf was not impaired in lysates from Semapimod-treated lipopolysaccharide-stimulated cells and Western blot analysis confirming that Semapimod treatment does not interfere with the lipopolysaccharide-dependent phosphorylation of this site (FIG. 4).

The present inventors have found that the MEK kinase c-Raf is a relevant target for Semapimod and consequently that c-Raf enzymatic activity is critical in Crohn's disease. The present inventors have also found that the use of array technology for kinome profiling is suitable for identifying specific biochemical changes in cellular metabolism. Kinome analysis is a useful and valuable method to determine the enzymatic activities of a large group of kinases.

The western blot experiments (4/4 macrophages; murine), demonstrate that Raf is the molecular target of Semapimod. The in vitro kinase data clearly shows that Raf is the molecular target of semapimod in 2 independent experiments, confirming the western blot data.

The immunohistochemistry (IHC) experiments, which includes blind scoring and analysis of the IHC results, demonstrate a significant decrease of pMek1/2 expression, which is localized to epithelial cells and lymphoid cells, in Semapimod treated Crohn's disease patients (n=5; all responders, e.g. CRP decrease). Biopsies obtained from the non-responder (n=1, pat.012) did not show a Semapimod-induced inhibition of pMek1/2 protein expression.

The effects of semapimod on cytokine synthesis were also studied in human cell types. Human CD4+ lymphocytes were isolated, and, via a “cell sorting procedure” CD4+ naive and CD4+ memory cells were obtained. Cells were LPS-stimulated and treated with/without 0.1 and 1 μM semapimod. Next week, supernatants (different time points) will be analyzed by CBA (Cytokine Bead Array) for cytokine expression.

The present invention makes it possible to interpret the data as that by using substrate arrays for the parallel determination of enzymatic activities. Heretofore, the metabolomics effort has been hampered by the lack of techniques that allow high-throughput analysis of cellular metabolism. Current mass-spectrometrical techniques concentrate on the static determination of metabolite levels rather as the enzymatic activity of the biochemical process leading to these levels.

Not only is the present array suitable for kinome profiling but it is also suitable for assaying cellular activity with respect to dephosphorylation, acylation, acetylation, ubiquination etc. Thus arraying for enzymatic activities provides metabolomics with the equivalent of the DNA array analysis for genomics with respect to the possibility to quickly obtain a comprehensive description of cellular metabolism and cellular transcriptome respectively. The invention has shown that kinome wide analysis of biologically relevant samples is a highly useful tool for studying the biochemical changes underlying cellular signal transduction.

Based on these findings, a person of ordinary skill in the art can incorporate a guanylhydrazone, and in particular Semapimod, into compositions and therapies for treating the known Raf mediated diseases and conditions.

The entire contents of each of the following references, and all references cited herein, are hereby incorporated by reference for all purposes, the same as if set forth at length.
1. Conway T. & Schoolnik, G. K., “Microarray expression profiling: capturing a genome-wide portrait of the transcriptome.” Mol. Microbiol. 47, 879-889 (2003).
2. Wurmbach, E. et al., “Validated genomic approach to study differentially expressed genes in complex tissues.” Neurochem. Res. 27, 1027-1033 (2002).
3. Velculescu, V. E. et al. “Analysis of human transcriptomes.” Nat. Genet. 23, 387-388 (1999).
4. Blom, N. Kreegipuu, A. & Brunak, S. “PhosphoBase: a database of phosphorylation sites.” Nucleic Acids Res. 26, 382-286 (1998).
5. Kreegipuu, A. Blom, N. & Brunak, S., “PhosphoBase, a database of phosphorylation sites: release 2.0.” Nucleic Acids Res. 27, 237-239 (1999).
6. Diks, S. H. van Deventer, S. J. & Peppelenbosch, M. P. Lipopolysaccharide recognition, internalisation, signalling and other cellular effects. J Endotoxin Res. 7, 335-48 (2001).
7. Morrison, D. C. & Ryan, J. L., “Bacterial endotoxins and host immune responses.” Adv. Immunol 28, 293-450 (1979).
8. Weinstein, S. L., Sanghera, J. S., Lemke, K. DeFranco, A. L. & Pelech, S. L., “Bacterial lipopolysaccharide induces tyrosine phosphorylation and activation of mitogen-activated protein kinases in macrophages.” J. Biol. Chem. 267, 14955-14962 (1992).
9. Monick, M. M. Carter, A. B. Flaherty, D. M. Peterson, M. W. & Hunninghake, G. W. “Protein kinase C zeta plays a central role in activation of the p42/44 mitogen-activated protein kinase by endotoxin in alveolar macrophages.” J Immunol 165, 4632-9 (2000).
10. Komalavilas, P. Shah, P. K. Jo, H. & Lincoln, T. M. “Activation of mitogen-activated protein kinase pathways by cyclic GMP and cyclic GMP-dependent protein kinase in contractile vascular smooth muscle cells.” J Biol Chem 274, 34301-9 (1999).
11. Ohmori, Y. & Hamilton, T. A. “Requirement for STAT1 in LPS-induced gene expression in macrophages.” J Leukoc Biol 69, 598-604 (2001).
12. Hommes, D. et al., “Inhibition of stress-activated MAP kinases induces clinical improvement in moderate to severe Crohn's disease.” Gastroenterology 122, 7-14 (2002).
13, Cohen, P. S. et al., “CNI-1493 inhibits monocyte/macrophage tumor necrosis factor by suppression of translation efficiency.” Proc. Natl. Acad. Sci. U.S.A. 93, 3967-3971 (1996).
14. Peppelenbosch, M. P. DeSmedt, M., ten Hove, T., van Deventer, S. J. & Grooten, J., “Lipopolysaccharide regulates macrophage fluid phase pinocytosis via CD14-dependent and CD-14-independent pathways.” Blood 93, 4011-4018 (1999).

### EXAMPLES

Having generally described this invention, a further understanding can be obtained by certain specific examples, which are provided herein for purposes of illustration only, and are not intended to be limiting unless otherwise specified.

### Materials and Methods I

### Chemicals

The catalytic subunit of Protein Kinase A was purchased from Promega. Semapimod (CNI-1493) was provided by Cytokine PharmaSciences, Inc.

### Peripheral Blood Mononuclear Cells Isolation

Peripheral Blood Mononuclear Cells (PBMCs) were isolated from peripheral blood from healthy volunteers using standard density-gradient centrifugation over Ficoll-Paque Plus (Amersham Pharmacia Biotech AB, Uppsala, Sweden) after washing in resuspension in IMDM supplemented with 10% FBS, penicillin, streptomycin and amphotericin (hereafter referred to as complete IMDM).

### Western Blot Analysis

For western blot samples, 106 PBMCs were suspended in 1 ml media and LPS stimulations were 15 minute incubations (37° C., 5% CO₂) with 10 ng/ml LPS or pre-incubated with 1.0 μM CNI-1493 (Cytokine PharmaSciences, Inc.). Stimulations were terminated by an ice-cold PBS wash. Cells were subsequently pelleted, lysed, denatured (5 minutes at 95° C.) and stored at −20° C. 10⁶ PBMCs lysed in 200 μl SDS Sample Buffer (62.5 mM Tris-HCl (pH 6.8 at 25° C.), 2% w/v SDS, 10% glycerol, 50 mM DTT, 0.01% w/v bromophenol blue), 25 μl of which was separated on 12% SDS-PAGE gels and transferred to nitrocellulose membranes. Antibodies for immunoblotting were from Cell Signaling and included Phospho-p38 MAP Kinase (cat #9211), Phospho-p44/42 MAP Kinase (cat #9101), Phospho-PKCzeta/lambda (cat #9378), Phospho-MEK1/2 (cat #9121), Phospho-Raf (cat #9424) and Phospho-SAPK/JNK (cat #9255). All antibodies were used in accordance with supplier's protocol and images were revealed with a Lumi-Imager (Boehringer Mannheim) using the chemoluminescence substrate Lumilight⁺ (Roche, Mannheim, Germany).

### PepChip Analysis

For kinome array samples, 10⁷ PBMCs were suspended in 5 ml media (IMDM supplemented with 1.0% human serum). LPS stimulation consisted of a minute incubation (37° C., 5% CO₂) with 100 ng/ml LPS (E. coli, serotype 0111:B4). CNI treated PBMCs were maintained in media dosed with 1.0 μM CNI-1493 (Cytokine PharmaSciences, Inc.) 1 hour before and during incubation for LPS stimulation. Stimulations were terminated by an ice-cold PBS wash. PBMCs were lysed in 200 μl cell lysis buffer (20 mM Tris-HCl (pH 7.5), 150 mM NaCl, 1 mM Na₂EDTA, 1 mM EGTA, 1% Triton, 2.5 mM sodium pyrophosphate, 1 mM MgCl₂, 1 mM beta-glycerophosphate, 1 mM Na₃VO₄, 1 mM NaF, 1 μg/ml leupeptin, 1 μg/ml aprotinin, 1 mM PMSF) and the volume of the cell lysate were equalised with dH₂O. The cell lysates were subsequently cleared on a 0.22 μm filter. PepChip incubation mix was produced by adding 10 μl of filter-cleared activation mix (50% glycerol, 50 μM ATP, 0.05% v/v Brij-35, 0.25 mg/ml BSA, ³³P-γ-ATP (1000 kBq)). Next, the PepChip mix is added onto the chip and the chip is kept at 37° C. in a humidified stove for 90 minutes. Subsequently, the PepChip was washed once with TBST, twice in 2M NaCl and twice in demineralised H₂O and air-dry the PepChip slips. The experiments were performed three times in duplicate. The average inter-experimental variance in relative values was less than 5% for all spots.

### Analysis of PepChip

The chips were exposed to a phosphoimager plate for 72 hours and the spots were analysed on AIDA analysing software. For graphical visualisation of the spots in FIG. 2 the data of four arrays was averaged to eliminate false positives.

TABLE 1

| Kinase | Substrate | Control | LPS | LPS + CNI* |
|---|---|---|---|---|
| Auto | PKG | 246 | 8100 | 320 |
| JAK | STAT1a/b | 248 | 7704 | 231 |
| Scr-family? | PTP-2C | 287 | 5960 | 250 |
| PKA/Ca2+ | Trp-5-MO | 1164 | 23925 | 1305 |
| CytoK Rec | FPS | 270 | 5512 | 265 |
| PKA | GS | 795 | 7980 | 340 |
| PKC | EIF4E | 4494 | 41272 | 6699 |
| PKA | v-Rel | 291 | 2574 | 291 |
| PKA | P450 | 2412 | 19980 | 265 |
| CKII | Lamin B R | 214 | 1736 | 250 |
| Cdc2 | CKII | 268 | 1728 | 265 |
| PKA | MBP | 280 | 1250 | 265 |
| CKII | TOPII | 231 | 900 | 900 |
| PKC | ACC-a | 2750 | 10692 | 861 |
| PKA | HSP27 | 288 | 1072 | 298 |
| CKII | Lamin D | 242 | 822 | 320 |
| CKI | GS | 5616 | 13386 | 300 |
| p38 | MAPKAP2 | 10168 | 21440 | 1150 |
| Src | Annexin2 | 4438 | 8745 | 287 |
| p34cdc2 | CKII | 320 | 560 | 291 |
| | GS | 9758 | 17052 | 24415 |
| PKC | BICKS | 23400 | 38086 | 15980 |
| PKA | MBP | 536 | 822 | 248 |
| v-Fps | Caldesmon | 16002 | 22800 | 31584 |
| | MBP | 26208 | 36442 | 51888 |
| PKC | c-RAF | 10764 | 13640 | 9522 |
| PKC | Trop | 18786 | 23230 | 40000 |
| | EIF4F | 21000 | 25200 | 24624 |
| p34cdc2 | AP-1 | 460 | 548 | 274 |
| | Vimentin | 26240 | 30889 | 59840 |
| | GS | 16920 | 19500 | 23664 |
| p34cdc2 | Vimentin | 192 | 214 | 300 |
| | MLC-2 | 48400 | 53482 | 33674 |
| p34cdc2 | c-Src | 230 | 248 | 270 |
| | MBP | 60630 | 62900 | 65076 |
| GSK3 | GS | 312 | 320 | 250 |
| CK II? | TOPII | 274 | 280 | 300 |
| PKA | Tau | 340 | 340 | 250 |
| | GS | 20736 | 20592 | 26477 |
| | GS | 31500 | 31050 | 51102 |
| PKC | c-Src | 10728 | 10416 | 17000 |
| PKC | HSP27 | 291 | 280 | 300 |
| | MBP | 38634 | 36464 | 38354 |
| GRK | Tau | 248 | 230 | 250 |
| PKA | MARCKS | 34295 | 31647 | 62514 |
| | MBP | 26481 | 23852 | 51414 |
| | MBP | 40793 | 36600 | 55572 |
| Lck | Tec | 18096 | 13824 | 300 |
| PKA | GS | 1440 | 1020 | 3450 |
| PKC | MARCKS | 23714 | 16401 | 13426 |
| | Tau | 15846 | 10500 | 27753 |
| CaM-II | Vimentin | 6930 | 4147 | 248 |
| PKA | c-Rel | 421 | 250 | 261 |
| p34cdc2 | Lamin B1 | 21000 | 11165 | 300 |
| | Vimentin | 37760 | 18020 | 44200 |
| CaM-II | MBP | 600 | 265 | 680 |
| p42/44 | MBP | 33814 | 14514 | 26352 |
| PKC | MBP | 47940 | 18624 | 15680 |
| CKII | GS | 17680 | 5350 | 13120 |
| Lck/Fyn | MHC-I | 7040 | 1370 | 230 |
| PKC | Lamin B1 | 2016 | 268 | 1072 |
| | | 4960 | 530 | 254 |
| CKII | TOPII | 3836 | 320 | 291 |
| PKC | MBP | 2376 | 198 | 319 |
| | c-Src | 34410 | 2470 | 319 |
| | | 16320 | 822 | 300 |
| p42/44 | MBP | 28710 | 536 | 23296 |
| PKA, p42/44 | CgA | 62088 | 548 | 6700 |

*CNI = CNI-1493 = semapimod

### Semapimod Mediated Effects In Vitro and In Vivo

To further elucidate the molecular target of Semapimod, the following experiments were carried out.

### Materials and Methods II

4/4 macrophages were used for in vitro experiments and western blot analysis. An in vitro kinase assay was performed to reconfirm findings obtained with western blotting.

Biopsies obtained from Crohn's disease patients (before and after Semapimod treatment) who participated in the Semapimod-pilot study in the AMC, were analyzed by immunohistochemistry.

The effect of Semapimod on cytokine synthesis in human CD4⁺ cells (naïve versus memory CD4⁺ cells) will be analyzed by CBA (Cytokine Bead Array).

### Results

### In Vitro Work

Raf is the molecular target of Semapimod.

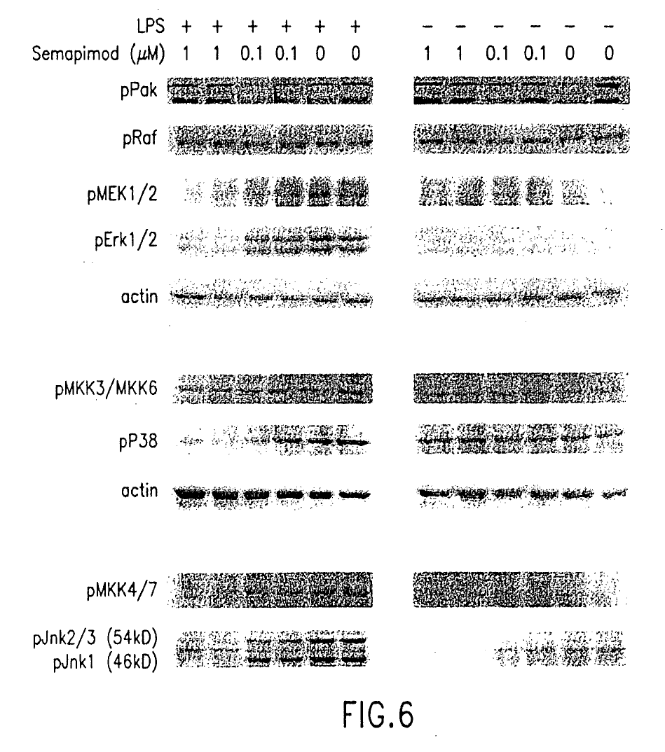

As previously shown, Semapimod concentrations≦1 μM did not have a cytotoxic effect in 4/4 macrophages (murine). 4/4 macrophages were grown to 70% confluence, pre-incubated for 1 hour with Semapimod (0.1 and 1 μM) and stimulated with LPS (100 ng/ml) for 15 minutes. Cells were harvested in sample buffer and whole cell lysates were loaded onto 10% SDS-PAGE and subsequently transferred to a PVDF membrane. Blots were stained with phospho-specific antibodies for various MAPK signaling molecules. FIG. 6 shows Semapimod-induced decrease of phosphorylated-P38, phosphorylated-Erk1/2 and phosphorylated-Jnk. Moreover, reduced expression of pMKK4/7 and pMek1/2-upstream kinases of Jnk and Erk respectively-, was observed upon Semapimod treatment. Importantly, pRaf protein levels and pPak, upstream of Raf, were not affected due to Semapimod incubation. Taken together, the in vitro data indicate that Raf is the molecular target of Semapimod.

FIG. 6 Western blot: Reduced pP38, pJnk, pErk in 4/4 macrophages treated with Semapimod (1 and 0.1 μM) in LPS present conditions was seen. Moreover, the phosphorylation status of MKK4/7 (upstream of Jnk), and Mek1/2 (upstream of Erk) was affected due to Semapimod treatment. pRaf expression was not influenced by Semapimod. To test for equal loading, western blots were analyzed for actin expression. Similar results were obtained in three independent experiments.

### Raf In Vitro Kinase Assay:

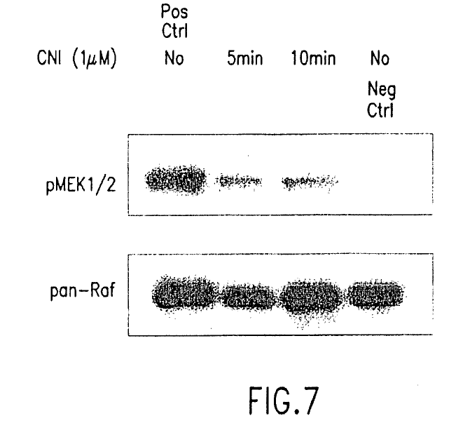

A commercially available in vitro kinase kit (Upstate, cat#: 17-360) was used to study the direct effect of Semapimod on Raf kinase activity. Active Raf kinase was incubated with 1 μM Semapimod for 5 and 10 minutes and subsequently inactive Mek-substrate was added. Furthermore, active Raf together with Mek, without Semapimod, served as a positive control. Active Raf without the Mek-substrate, left untreated, served as a negative control. The in vitro kinase reaction was performed at 30 C.° for 20 minutes. Sample buffer was added to terminate the reaction, and loaded on 110% SDS-PAGE. The immunoblot was analyzed for pMek expression. These data indicate that Raf kinase activity is inhibited by Semapimod. The results are shown in FIG. 7.

FIG. 7 Western blot: in vitro kinase assay showed reduced pMek1/2 expression upon 5 and 10 minute treatment with 1 μM Semapimod. Pan-Raf protein expression was analyzed to test for equal loading. Two experiments were performed and similar results were obtained.

### Immunohistochemistry

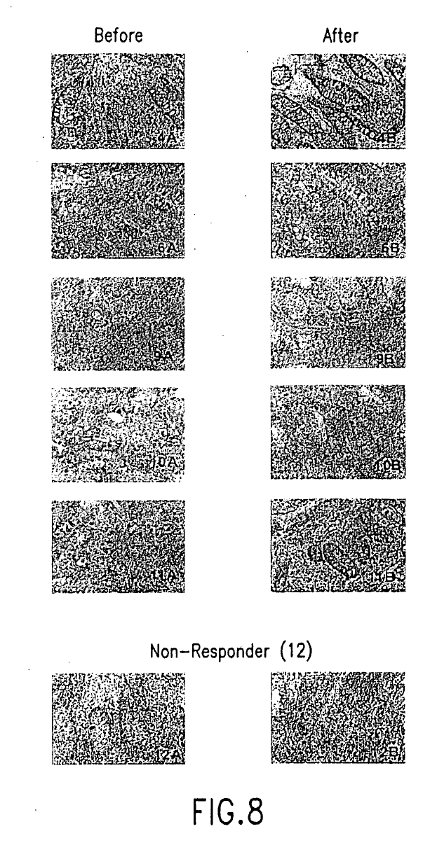

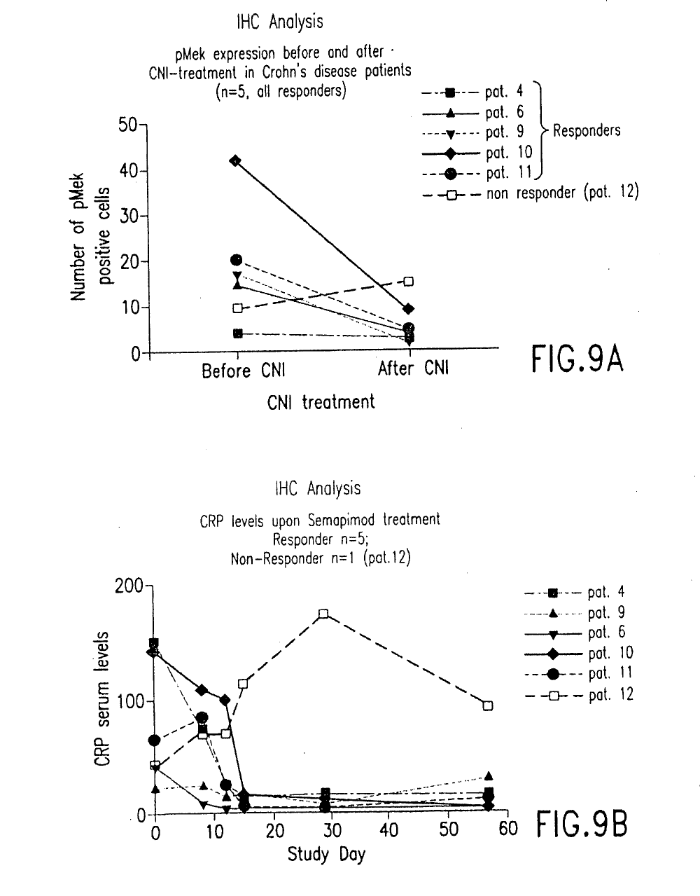

Biopsies obtained from Crohn's disease patients (before and after treatment) who participated in the Semapimod-pilot study performed in our department, were analyzed for pMek1/2 expression (the substrate of Raf) by immunohistochemistry (FIGS. 8-9). These data demonstrate that pMek1/2 expression is localized to intestinal epithelial cells and lymphocytes. Importantly, a significant decrease (p=0.0348) of pMek1/2 expression in Semapimod treated patients who responded to Semapimod (n=5) was seen, shown in FIG. 9. Immunohistological staining of tissue obtained from one nonresponder (e.g. no decreased CRP levels), showed increased pMek1/2 expression after Semapimod treatment. These data link the Raf kinase to Crohn pathology.

FIG. 8 shows the immunohistochemical staining of biopsies obtained from Crohn's disease patients who participated in the Semapimod study. In FIG. 8, the immunohistochemical staining of biopsies obtained from responders (n=5) demonstrates a significant decrease (p=0.0348) of pMek1/2 expression upon Semapimod treatment. FIGS. 9A and B show the IHC analysis of Crohn's disease patients who participated in the Semapimod pilot study. FIG. 9A generally shows the pMek expression before and after CNI-treatment in Crohn's disease patients, and FIG. 9B generally shows the CRP serum levels of these patients. CRP levels of patient #12 were not affected by Semapimod (e.g. nonresponder). Importantly, pMek1/2 expression was also not affected by Semapimod in this nonresponder. As shown in FIG. 9A, very little pMek1/2 expression was seen at baseline in patient 004. This finding correlates with the low CRP levels at baseline (FIG. 9B).

### Cytokine Analysis

CBA: Human naïve and memory CD4⁺ cells were isolated from a healthy volunteer and treated with Semapimod (1 μM and 0.1 μM) or left untreated. Subsequently, cells were stimulated with LPS (100 ng/ml) and kept in the incubator at 37 C.°. Supernatant was harvested at different time points and stored at −20 C.° for CBA analysis. A CBA will be performed for IL-1β, TNFα, IL-6, IL-8, IL-12p70 and IL-10 cytokine levels (human inflammation kit).

The present inventors have found that Raf is the molecular target of Semapimod. The in vivo data suggest that Raf is an important player in the pathogenesis of Crohn's disease.

Obviously, numerous modifications and variations of the present invention are possible in light of the above teachings. It is therefore understood that within the scope of the appended claims, the invention may be practiced otherwise than as specifically recited herein.

1. A method for the treatment of a condition mediated by raf kinase, comprising administering a guanylhydrazone to a subject in need thereof.

2. The method according to claim 1, wherein the condition is selected from the group consisting of cancer, histiocytic lymphoma, lung adenocarcinoma, small cell lung cancer, pancreatic carcinoma, breast carcinoma, thyroid carcinoma, murine cancer, bladder carcinoma, colon carcinoma, myeloid disorder, myeloid leukimia, villous colon adenoma, a disorder associated with ischemic-event-induced neoronal degeneration, cardiac-arrest-induced cerebral ischemia, stroke-induced cerebral ischemia, multi-infarct-dementia-induced cerebral ischemia, head-injury-induced-cerebral ischemia, surgery-induced cerebral ischemia, childbirth-induced cerebral ischemia, Crohn's disease, and a combination thereof.

3. The method according to claim 1, wherein the guanylhydrazone is Semapimod.

4. The method according to claim 1, wherein the subject is a human.

5. The method according to claim 1, wherein the condition is mediated by a process comprising a modulation or inhibition of signal transduction through the c-Raf kinase.

6. The method according to claim 1, wherein the condition is mediated by a process comprising a modulation or inhibition of signal transduction through the c-Raf MEK kinase.

7. The method according to claim 1, wherein the raf kinase is c-raf kinase.

8. The method according to claim 1, wherein the raf kinase is c-raf MEK kinase.

9. A method, comprising modulating or inhibiting signal transduction in a c-raf pathway with at least one guanylhydrazone.

10. The method according to claim 9, wherein the guanylhydrazone is Semapimod.

11. A method, comprising:
contacting one or more human mononuclear cells with at least one guanylhydrazone and at least one lipopolysaccharide to obtain one or more treated cells;
contacting at least one selected from the group consisting of said treated cells, one or more lysates thereof, and combinations thereof, with at least one surface-bound peptide in a surface-bound peptide array; and
selectively modulating or inhibiting the phosphorylation of the surface-bound peptide.

12. The method according to claim 11, wherein the guanylhydrazone is Semapimod.

13. The method according to claim 11, wherein the surface-bound peptide comprises at least one consensus phosphorylation sequence for MAP kinase or MEK kinase.
